(19) United States
(12) Patent Application Publication
(10) Pub. No.: US 2021/0138017 A1
(43) Pub. Date: May 13, 2021

(54) Kravinol

(71) Applicant: Christopher Shane Chaffins, Lexington, KY (US)

(72) Inventor: Christopher Shane Chaffins, Lexington, KY (US)

(21) Appl. No.: 16/678,309

(22) Filed: Nov. 8, 2019

(51) Int. Cl. A61K 36/74; A61K 36/67; A61K 31/05; A61K 31/352; A61P 25/36; A61P 25/32

(52) U.S. Cl. CPC A61K 36/74; A61K 36/67; A61P 25/32; A61K 31/352; A61P 25/36; A61K 31/05

(57) ABSTRACT

A medication/supplement with four components/parts/supplements/ingredients which are kratom, kava, CBD (cannabidiol) and THC (tetrahydrocannabinol) that are combined and produced in a pharmaceutical company/corporation/entity/nutritional company/corporation/factory in any properly adjusted amount for best practices in treating individuals with alcohol and/or drug addiction/cravings.

### FIELD

The present application relates to 4 natural components/ingredients added together in best practices format medically/supplementary to treat cravings for alcohol and/or drugs in those individuals with addiction/substance abuse problems/issues.

### BACKGROUND

Natural substances and ingredients are increasingly showing more favorable results in subjects over synthetically produced drugs due to a more naturally occurring mechanisms and pharmacodynamics. The ingredients used for such supplements and dietary compounds have scientifically been tested and proven to show benefit/s in a variety of subjects with differentiated diagnoses.

Companies/Corporations/Scientists/Labs/Doctors/Medical Staff are trying to create a medication that adheres more to the naturalistic approach without causing the damage synthetically produced medication/s produce in a person's body.

### SUMMARY

By providing a medication/supplement from natural components/ingredients, a person's risk to internal damage or replacing one addiction with another is significantly reduced.

The disclosed mixture takes 4 basic naturally occurring components/parts/supplements/ingredients and provides a medication and/or supplement to effectively treat alcohol and/or drug cravings after a person has detoxed successfully from alcohol and/or drugs in a medical rehab facility/hospital.

The mixture is made up of four components/parts/supplements/ingredients. These ingredients combined are more naturally occurring and are designed to curve and/or stop cravings for alcohol and/or drugs (opioids/benzodiazepines for example).

### BRIEF DESCRIPTION OF THE MEDICATION/SUPPLEMENT

There are no drawings to display, however there is an explanation with reference to the four components/parts/supplements/ingredients (listed below) which makes up Kravinol:
Ingredient 1—Kratom
Ingredient 2—Kava
Ingredient 3—CBD (cannabidiol)
Ingredient 4—THC (tetrahydrocannabinol)

### DETAILED DESCRIPTION

The following are two different dosages of the medication/supplement. The aforementioned herein 1.0 and 2.0 doses may be changed by a pharmaceutical company/corporation/entity/nutritional company/corporation for best practices in relation to patient care using the independent components (kava, kratom, CBD, THC) to make the best version of the dependent component “Kravinol.” This drug/supplement is designed to target/prevent drug/alcohol cravings/relapse rather than to fix/inhibit early withdrawal symptoms the Subject/Patient may be experiencing or prevent death during severe withdrawal symptoms from drugs and/or alcohol in which some cases naloxone should be administered. It will be up to the pharmaceutical company/corporation/entity/nutritional company/corporation how much the aforementioned independent components (kava, kratom, CBD, THC) should be changed to yield the best version of the dependent component “Kravinol.”

### Kravinol 1.0

Each Gel Capsule/Capsule/Tablet/Supplement Contains—500 mg of Kratom, 500 mg of Kava, 500 mg of CBD (cannabidiol), 10 mg of THC (tetrahydrocannabinol).

### Kravinol 2.0

Each Gel Capsule/Capsule/Tablet/Supplement Contains—1000 mg of Kratom, 1000 mg of Kava, 1000 mg of CBD (cannabidiol), 20 mg of THC (tetrahydrocannabinol).

We understand Subjects/Patients may need to remain on their antidepressant medication/s and/or other non-addictive prescription drug's and will need to gradually decrease and eventually stop their benzodiazepine/opioid intake to prevent death. It is not known whether or not Kravinol interferes with the function of these medications within the brain/body. The following are two different doses of Kravinol. The manufactured dose, however can be adjusted according to the pharmaceutical company/corporation/entity/nutritional company/corporation with additional input from Physicians/Medical Directors for best practices which yields the best version. Any pharmaceutical company/corporation/entity/nutritional company/corporation shall/can adjust each independent component (kava, kratom, CBD, THC) accordingly to make/produce the best version of the dependent component “Kravinol.” Kravinol can be made in a pharmaceutical company/corporation/entity/nutritional company/corporation factory by combining the following components/parts/supplements/ingredients together.
Ingredient 1—Kratom
Ingredient 2—Kava
Ingredient 3—CBD (cannabidiol)
Ingredient 4—THC (tetrahydrocannabinol)

1. A medication/supplement wherein composed of kratom, kava, CBD (cannabidiol) and THC (tetrahydrocannabinol) to treat alcohol and/or drug (opioids/benzodiazepines for example) cravings in those individuals suffering from alcohol and/or drug addiction/cravings.
